(19) United States
(12) Patent Application Publication
(10) Pub. No.: US 2026/0022327 A1
(43) Pub. Date: Jan. 22, 2026

(54) DEVICE AND METHOD FOR AUTOMATIC CELLULAR SORTING AND ANALYSIS VIA ROBOTIC MOTOR-DRIVEN FLOW

(71) Applicant: THE REGENTS OF THE UNIVERSITY OF CALIFORNIA, Oakland, CA (US)

(72) Inventors: Dino Di Carlo, Los Angeles, CA (US); Reem Khojah, Los Angeles, CA (US)

(73) Assignee: THE REGENTS OF THE UNIVERSITY OF CALIFORNIA, Oakland, CA (US)

(21) Appl. No.: 18/993,513

(22) PCT Filed: Jul. 11, 2023
(86) PCT No.: PCT/US2023/069982

§ 371 Date: Jan. 10, 2025

Related U.S. Application Data
(60) Provisional application No. 63/388,595, filed on Jul. 12, 2022.

(51) Int. Cl. C12M 1/00; F15D 1/10

(52) U.S. Cl. CPC C12M 47/04; F15D 1/10

(57) ABSTRACT

Automating operations and handling of mesoscale biological objects that range in size from ˜100 μm to ˜1 mm can accelerate the advancement of disease modeling and drug discovery based on organoids and cell-carrier systems. The device leverages flow driven by rotating shafts in a Stokes flow regime to develop a gentle robotic manipulation system for objects based on controllable arrays of micro-motors. The underlying motor-flow mechanics was investigated that enable linear flow control from one pair of motors to an ensemble of reconfigurable motors. This robotic motor-flow driving system can operate in standard cell culture containers (e.g., Petri dish) to perform precise and real-time control of mesoscale cellular constructs in diverse biocompatible carrier fluids such as cell media. Using optional image-based feedback, a fully automated bioparticle sorting system may be created based on the device.

### RELATED APPLICATION

This Application claims priority to U.S. Provisional Patent Application No. 63/388,595 filed on Jul. 12, 2022, which is hereby incorporated by reference. Priority is claimed pursuant to 35 U.S.C. § 119 and any other applicable statute.

### TECHNICAL FIELD

The technical field generally relates to devices and methods for performing automated operations and handling of mesoscale biological objects that range in size from ˜100 μm to ˜1 mm. In particular, the technical field relates to a device and method that uses rotating shafts in a Stokes flow regime to robotically manipulate biological objects based on controllable arrays of micro-motors that drive the shafts.

### BACKGROUND

Automated manipulation and sorting of bio-particles in fluids via liquid handling robotics can support future AI-driven biomedical laboratories. In recent years, cellular aggregates, clusters, organoids, and biomaterial-cell constructs are playing a significant role in understanding physiology and testing drugs while incorporating more physiological relevance than single cells. These intermediate size bioparticles (˜100 μm to ˜1 mm) enable a range of biomedical applications from directed cell population evolution via hydrogel droplets or particles with 100-500 μm diameters, to mimicking in-vivo organs in drug screening via spheroids, embryoid bodies, organoids and assembloids with 100 μm-1 mm diameters. However, it is challenging to automate flow manipulation of mesoscale particles because they sit in a gap between commercial flow manipulation technologies (FIG. 1A); microfluidic and flow cytometry technologies specifically designed for microscale particles and single cells and robotic liquid handling technologies designed for milli-scale particles and reaction volumes. Mesoscale bioparticles are too small and may be lost in marginal pipetting errors of robotic milli-liquid handling technologies. At the same time, mesoscale bioparticles are too big to have negligible inertia and may clog microfluidic channel flows or flow cytometers. This wide size range requires a modular fluidic manipulation system to accommodate linear flow control for a range of mesoscale particles sizes and shapes. Lastly, their complex structures require high-content screening methods like image-based analysis prior to sorting. Therefore, these newly developed mesoscale bioparticles necessitate a scalable and modular flow control approach to enable automated manipulation, image-based analysis, and multi-tasking in unconfined conditions (e.g., a Petri dish, well plate, or multi-well plate).

### SUMMARY

To scale the manipulation of mesoscale bioparticles in fluid, a device for the control of objects within unconfined conditions was developed to mechanically drive open flow of a viscous biocompatible fluid via arrays of computer-controlled stepper micromotors. These embedded stepper micromotors were programmed to automatically position and sort particles at the micro and milli scale. The device or system is referred to herein as micromotor-driven millifluidics (MDM). First, Jeffery's 1922 analytical solution of flow between two rotating circular cylinders or motors was numerically and experimentally expanded on. When the two circular motors rotate in opposite directions (i.e., clockwise and anti-clockwise), they form a unidirectional flow in between, resembling channel flow but without confining walls. Since Jeffery's flow is tunable by changing motor size, speed, rotation direction, and the gap between motors, the approach is able to produce many of the same operations of channel-based microfluidic flows without the need of microfabrication or the use of external pumps. Arrays of reconfigurable and synchronized microscale robotic motors (e.g., used for flying drones), which were not available at the time of Jeffrey's analytical model was developed, were then introduced in an open Petri dish because it provides a platform for the culture and imaging of most mesoscale cellular structures. This device enabled modular and programmable mesoscale flow using linear closed-loop control and sort polydisperse droplets based on the encapsulated cell number. This fully automated motor-driven flow control system enables future biomedical automated experimentation powered by computer intelligence to supervise mesoscale bioparticle analysis, sorting and manipulation.

Precise and instant control of suspended particles benefits from a reproducible and non-computationally demanding linear closed-loop system with real-time feedback between the computer controller and the fluid manipulation system (FIG. 6A). In such linear control systems, suspended particles respond to the fluid flow in a predictable manner and precisely move to the target location set by the controller (FIG. 1D).

In one embodiment, a device for the control of objects in a fluid within unconfined conditions is disclosed. The device includes a plurality of rotatable motor shafts arranged in an array or pattern and in physical contact with the fluid containing the objects, the plurality of rotatable shafts coupled to respective motors. The device includes a controller or computing device operatively coupled with each of the respective motors and configured to control one or more of rotational direction and rotational speed of the plurality of rotatable shafts. The device may include an imaging device that captures images of the objects and image analysis software that is configured to track the spatial location and angular rotation of the objects. The image analysis software may also be used to identify the type or class of object and is used for sorting of the same.

In another embodiment, a method of manipulating objects in a fluid within unconfined conditions includes providing a plurality of motor-driven rotatable shafts in contact with the fluid containing the objects and controlling the rotational speed and/or direction of the plurality of rotatable shafts to controllably manipulate the objects within the fluid. Manipulation may include rotation and translation of the objects. The objects may be automatically translated to one or more target location based on the type or class of the objects. For example, cells of a particular phenotype can be automatically detected and sorted. In another example, droplets containing cell(s) may be detected and sorted based on the number and/or types of cells contained therein.

### BRIEF DESCRIPTION OF THE DRAWINGS

FIG. 1A schematically illustrates the gap that exists for mesoscale bioparticle manipulations. Mesoscale bioparticles sit in a gap between two developed technologies; microfluidics and liquid handling robotics for microscale and milli-scale particle manipulation, respectively.

FIG. 1B schematically illustrates a device for the motor-driven flow control of mesoscale particles. The device enables robotic mesoscale particle manipulation in any fluid container (e.g., Petri dish) by combining precise continuous flow manipulation features from microfluidics and linear control feedback automation features.

FIG. 1C illustrates a plurality of rotatable motor shafts operating in a fluid within unconfined conditions (e.g., Petri dish). Linear motor-driven flow in a number of different directions can be accomplished by adjusting the rotational direction and/or the rotational speed of the rotatable shafts or cylinders. FIG. 1C also illustrates an enlarged view of two rotating shafts or cylinders.

FIG. 1D illustrates the theoretical limitations of mesoscale particle control in confined channel flows due to competing inertial forces that prevent linear/reversible flow control.

FIG. 2A illustrates a schematic diagram of two counter rotating cylindrical motors with radius (a) rotating in a carrier fluid separated from the mid-plane between the motors by distance (e) from the motor wall and distance (L) from the motor center.

FIG. 2B illustrates streamlines of fluid flow between counter rotating motors like that of FIG. 2A from Jeffrey's flow analytical solution.

FIG. 2C illustrates the normalized vertical velocity (U_{y}) over the motor velocity (uₘ) at the centerline or mid-plane for the motor configuration of FIG. 2A. Validation of computed solution of motor-driven flow that matches Jeffery's flow analytical solution (1922).

FIG. 2D illustrates a plot of the distances between upstream and downstream saddle points (sp) as a function of the gap distance e.

FIG. 2E schematically illustrates that modular unidirectional channel flow with different lengths and widths between the motors that is reconfigurable for microscale particles.

FIG. 2F schematically illustrates that modular unidirectional channel flow with different lengths and widths between the motors that is reconfigurable for milliscale particles.

FIG. 3A illustrate a phase diagram of the flow topology between counter rotating motors as a function of Reynolds number (Re) and the non-dimensional gap distance between the motors (h). Three flow types are (◯) symmetric Jeffery's flow, (Δ) upstream and downstream jet flow, and (·) downstream jet flow.

FIG. 3B illustrates the characterization of motor-driven flow via flow tracing experimental results (left) and numerical simulation results (right).

FIG. 3C illustrates the motor-driven flow velocity profile which shows symmetry in Jeffery's flow.

FIG. 3D illustrates the motor-driven flow pressure profile which shows symmetry in Jeffery's flow.

FIG. 3E schematically illustrates how the flows with symmetry allow particle hand offs between upstream and downstream motor units which enables synchronizing functions in an ensemble of spatially-distributed and temporally sequenced motors.

FIG. 4A schematically illustrates how unconfined unidirectional flow by an ensemble of motor-flow driving units resemble channel flow in microfluidic devices.

FIG. 4B shows the unidirectional flow velocity (Ut) is connected between downstream lateral motor units for distances between motors pairs below D=3sp (lines of 1 sp, 2sp, 3sp). The 4sp, 5sp, 6sp lines indicate a reversing flow of U) which does not allow the flow to pass from upstream to downstream motor pairs.

FIG. 4C schematically illustrates how spatially-reconfigurable motors are used to design different shapes of pseudo channels keeping the maximum inter-motor distance (D). Simulation results of the motor flow velocity illustrates vertical flow directions and magnitudes for a pseudo channel containing a bend.

FIG. 4D illustrates microscope imaging of a fluorescent dye introduced into the motor-driven flow driven in a Petri dish which was used to illustrate the flow shape at the microscale (on the left) and a red food dye was used to illustrate the flow shape at the milli-scale (on the right) matching the same simulation results of the motor ensemble (FIG. 4C).

FIG. 5A schematically illustrates how a computer and/or controller is used to control a quadrupole ensemble of motors for flow control. The control may include the direction of rotation and speed of rotation.

FIG. 5B illustrates the programmable flow manipulation to vertical, horizontal, and diagonal directions by modulating rotation orientation (+/−S) to certain motors (Mn) of motors in a quadrupole arrangement.

FIG. 5C illustrates the simulation results of the motor vertical (U_{y}) and horizontal (Uₓ) velocity field to drive the flow in different directions with same motor rotations of FIGS. 5A and 5B.

FIG. 5D illustrates microscopic image snapshots of videos showing programmed particle manipulation in different directions via motor-driven flow. Movement direction is indicated by the arrows.

FIG. 5E schematically illustrates undefined modular motor flow control in a plurality of directions that uses a quadrupole ensemble of motors.

FIG. 5F illustrates programming motor flow control with a joystick or other input device that enables direct flow manipulation to achieve the flow modularity.

FIG. 6A illustrates diagram of the system for linear closed-loop control of motor-driven flow control or objects via image-based feedback.

FIG. 6B schematically illustrates a diagram of automated analysis and sorting of mesoscale droplets (i.e., objects) based on the number of encapsulated cells per droplet (0, 1, or 2).

FIG. 6C illustrates the programmable motor flow profiles used to sort the droplets that contain cells. Droplets are moved down, rotated, moved left, and moved right depending on the contents of the droplets.

FIG. 6D illustrates the automated droplet sorting using a continuous image-based feedback system. Automated sorting tasks and processing of a wide range of mesoscale droplet sizes and shapes is possible. Image analysis code performs the first task (1) to locate mesoscale particles in the Petri dish and continuously communicate their spatial coordinates (x,y) to the computer controller. Next, (2) a motor quadrupole rotates the droplets while the image recognition system analyzes and counts the number of cells in the droplet at different viewing angles. Lastly, (3) automated motor-driven sorting was performed to different target locations (e.g., outlets) based on the encapsulated cell number.

FIG. 7A shows bipolar coordinate system diagram of two co-axial motor-driven flow in bipolar coordinates.

FIG. 7B illustrates two identical cylindrical motors with slip boundary condition at the co-axial circles ξ₁ and ξ₂ in the (x,y) plane.

FIG. 7C illustrates the range of quasi-radial coordinates (−∞<ξ<+∞) and the angular coordinates (0<η<2π) in the bipolar coordinate system.

FIG. 8A illustrates the experimental setup of motor-driven flow in an open Petri dish with fluorescent particle tracers in the carrier fluid. The experimental setup includes counter-rotating motors.

FIG. 8B illustrates stitched microscopy images of fluorescence tracer particles for flow driven in a Petri dish tracing streamlines around two counter rotating motors.

FIG. 9 illustrates unidirectional flow as a function of the gap (E) between the two counter-rotating motors. The color scale indicates the vertical flow velocity magnitude (U_{y}) to illustrate the flow direction around the motors.

FIG. 10 illustrates flow profiles between counter-rotating motors as a function of the motor velocity (U_{θ}) from 2D simulations match the experimental results of flow characterization using fluorescent tracer beads. The color scale indicates the normalized fluid velocity (U_{y}/U_{θ}) to illustrate the flow direction at different motor velocities.

FIG. 11 illustrates motor-driven flow profiles as a function of Reynolds number illustrates three motor-driven flow profiles summarized in the phase diagram from FIG. 3A. Flow starts symmetrically, resembling Jeffery's flow. Flow becomes symmetrical at higher Reynolds number, with a majority of flow driven upstream of the motors. Then flow is driven downstream of the motors at even higher Reynolds numbers where Uv is the motor velocity and U is the fluid velocity magnitude.

FIG. 12A illustrates the scaling motor-driven flow with an ensemble of three pairs of motors to recreate the same unidirectional channel flow (channel flow on left).

FIG. 12B illustrates that an ensemble of motors in simulations drives flow in a similar manner to experiments, forming a unidirectional flow conduit that can transport a bead or other object.

FIG. 12C illustrates an image that tracks particle transport in the pseudo-channel that is formed.

### DETAILED DESCRIPTION OF ILLUSTRATED EMBODIMENTS

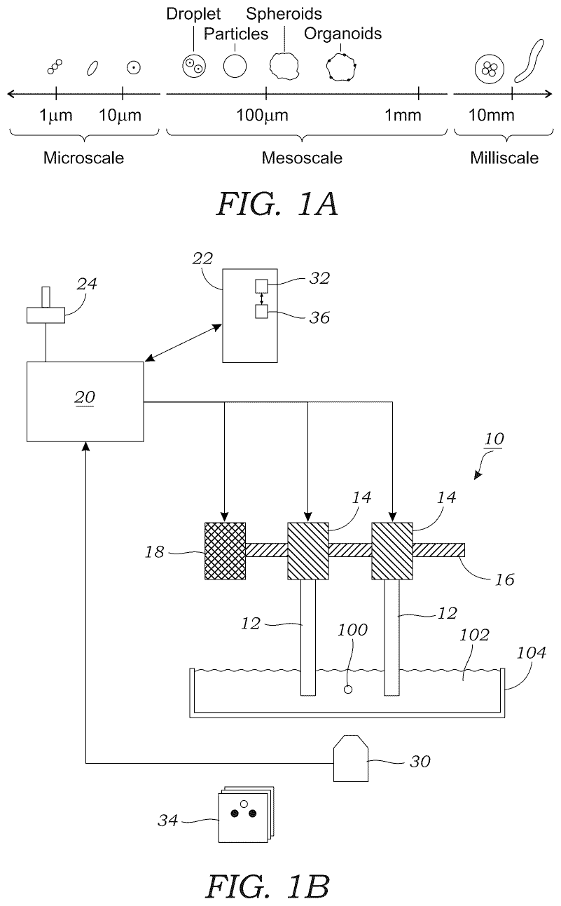

In one embodiment, and with reference to FIG. 1B, a device 10 is provided for the control of objects 100 located in a fluid 102 within unconfined conditions. The term “unconfined conditions” refers to the fact that the objects 100 in the fluid 102 are not contained within a defined flow path like a channel. For example, unconfined conditions exist when objects 100 are contained in a fluid 102 that is contained within fluid holder 104 such as a container, dish (e.g., Petri dish), or well such as that illustrated in FIG. 1C. There are no local flow constraints like microfluidic channel walls or the like. The objects 100 may include droplets, cells, cellular constructs, beads, cluster of cells, organoids, spheroids, or other particles. The objects 100 may range in size from ˜100 μm to ˜1 mm. In one embodiment, the object 100 that is to be manipulated is a droplet that contains one or more other objects 100 (e.g., cells or particles). The fluid 102 may include any biocompatible fluid. The fluid 102 may include an aqueous fluid or an oil-based fluid. When the objects 100 include droplets or droplet-like structures, the droplets can be made from an aqueous fluid, an oil-based fluid, or a hydrogel. Typically, the carrier fluid for the droplets is generally immiscible with the droplet fluid/material.

The device 10 includes a plurality of motor-driven rotatable shafts 12 arranged in an array or pattern and in physical contact with the fluid containing the objects 100. In one embodiment, there are at least four (4) rotatable shafts 12. For example, the rotatable shafts 12 may be arranged in a quadrupole configuration although other configurations are contemplated. However, in other embodiments there may be as few as one rotatable shaft 12. For example, a single rotatable shaft 12 may be used to rotate an object 100.

The diameter of the rotatable shafts 12 may vary from about 500 microns to several millimeters. The height and cross-sectional shape of the rotatable shafts 12 may vary. During use, the rotatable shafts 12 should extend into the fluid containing the objects 100 at least partially. The depth of penetration may vary but should be a majority of the overall fluid depth of the fluid holder 104. In some embodiments, the rotatable shafts 12 should extend substantially all of the depth of the fluid contained in the fluid within the fluid holder 104. The ends of the rotatable shafts 12, however, should avoid contact with the bottom of the fluid holder 104. The rotatable shafts 12 may be made from a number of materials including, for example, polymers, plastics, and metals. The rotatable shafts12 should be made from a material that is biocompatible with the objects 100 within the fluid. The rotatable shaft 12 should generally be formed to have a smooth outer surface that makes contact with the fluid. In one embodiment, the rotatable shafts 12 have a cylindrical shape.

Respective motors 14 are coupled to each of the rotatable shafts 12 and rotates the shafts 12 about their axis. The motors 14 may directly drive the rotatable shafts 12 or indirectly through a gears or other transmission. For example, in one embodiment, the rotatable shafts 12 are the actual shaft of the motor 14. The motors 14 and the rotatable shafts 12 may be contained in a fixture or rig 16 as seen in FIG. 1B that maintains a fixed arrangement of the rotatable shafts 12 relative to one another (e.g., rotatable shafts 12 arranged in a quadrupole configuration). The fixture or rig 16 may be coupled to moveable stage 18. The moveable stage 18 may move the fixture or rig 16 and/or the rotatable shafts 12 in the vertical (z) direction and/or the horizontal (x, y) direction 12. For example, the moveable stage 18 may lower the rotatable shafts 12 into the fluid 102 containing the objects 100. For instance, the rotatable shafts 12 can be moved into contact with the fluid 102 containing the objects 100 when used and can be removed following use. The positioning of the rotatable shafts 12 may also be adjusted in the horizontal direction as well.

A controller 20 is operatively coupled with each of the respective motors 14 and controls one or more of the rotational direction and/or the rotational speed of the plurality of rotatable shafts 12. Movement may include counter rotation of adjacent rotatable shafts 12 as described herein (e.g., one rotatable shaft 12 rotates in a clockwise direction while adjacent rotatable shaft 12 rotates in counter-clockwise direction). The controller 20 may contain instructions or a program that is executed or performed to manipulate the objects 100 in a pre-defined manner. The controller 20 may interface with a computing device 22 such as a computer that can be used to create instructions or programs that are then transferred to the controller 20. The controller 20 may also be integrated into the computing device 22 in other embodiments. That is to say, the controller 20 functionality may be incorporated into the computing device 22. In some embodiments, the controller 20 (or computing device 22) may be coupled to an input device 24 (e.g., like a joystick) to enable manual manipulation of objects 100.

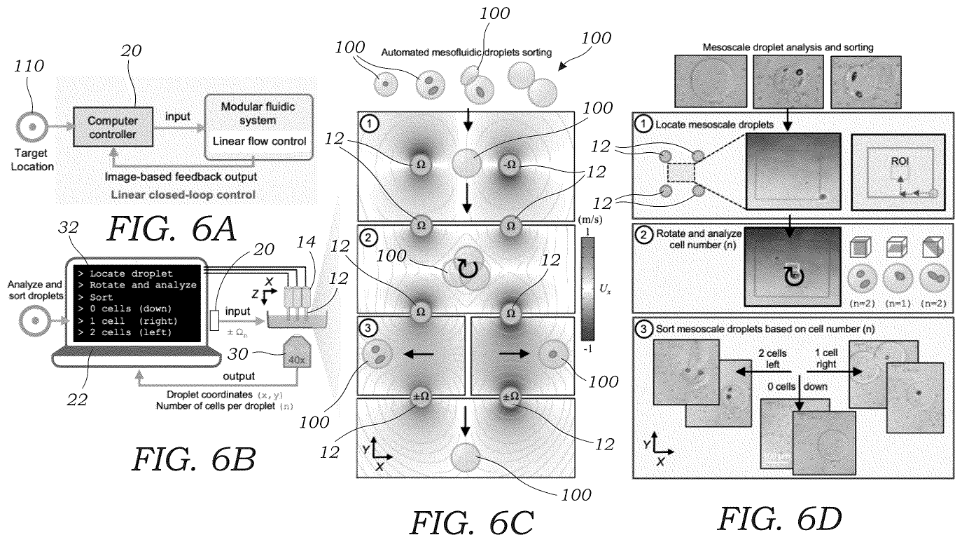

Manipulation may include rotation and/or translation of objects 100 within the fluid 102. Movement may include translations in any number of directions. In addition, in certain embodiments, reverse manipulation may take place in which the objects 100 substantially retrace to movements previously made. The controller 20 or computing device 22 may contain instructions to move the objects 100 to one or more target location(s) 110 as seen in FIG. 6A. The target location(s) 110 may include certain locations or regions within the fluid holder 104. The target location(s) 110 may include holding regions that temporarily store particular objects 100 that belong to a particular type or class. The target location(s) 110 may also include ports or outlets in the fluid holder 104 that may be used to remove the objects 100 from the fluid holder 104. The target location(s) 110 may be used to sort the objects 100 as described herein.

In some embodiments and with reference to FIG. 1B, an imaging device 30 such as microscope or the like is used with the device 10 for imaging of the objects 100. The imaging device 30 may further include image analysis software 32 that is used to track and identify the type or classes of objects 100. For example, the objects 100 in the fluid 102 are captured in images 34 obtained by the imaging device 30 and the image analysis software 32 may continuously communicate their spatial coordinates (e.g., x, y position within the fluid holder 104) to the controller 20 or computing device 22. The controller 20 or computing device 22 is configured to control each of the respective motors 14 in response to the location of the objects 100. The image analysis software 32 may also be used to identify the type or class of object 100. In some embodiments, artificial intelligence or a trained neural network or algorithm 36 (FIG. 1B) may be used for this task. For example, the image(s) of the objects 100 may be run through a trained neural network 36 or other machine learning to segment the objects 100 or identify the class or type of object 100. The image analysis software 32 may be used to count the number of cells contained within a droplet (i.e., object 100) or organoid as explained herein. Based on the number of cells within the droplets or organoid, the droplets or organoid can then be sorted to their target locations 110. Of course, other non-droplet objects 100 are also contemplated.

In some instances, the configuration of the rotatable shafts 12 may be adjusted or tuned. This includes altering the dimensions of the rotatable shafts 12, adjusting the spacing between the rotatable shafts 12, altering the number of rotatable shafts 12, and adjusting the pattern or arrangement of the rotatable shafts 12. The arrangement of rotatable shafts 12 may also be adjustable on-the-fly using the moveable stage 18 or other positioning instruments with tens of micrometer positioning accuracy. Adjusting of the rotational speed and direction of the rotatable shafts 12 is performed using the controller 20 or computing device 22. During use, at least one of the rotatable shafts 12 drives flow within the fluid 102. The flow of fluid 102 is in a laminar flow regime. In a preferred embodiment, the flow of fluid 102 created by the rotating shaft(s) 12 is at a Reynolds number of less than 10. In another embodiment, the flow of fluid 102 is at a Reynolds number that is less than 5. In still other embodiments, the flow of fluid is at a Reynolds number <<1. In some embodiments, flow conditions are created such that objects 100 of different sizes and/or shapes can be moved or translated to the same target location 110. In addition, in some embodiments, movement or translation may be reversed along substantially the same path.

### EXPERIMENTAL

### Results and Discussion

### Linear Motor-Driven Flow

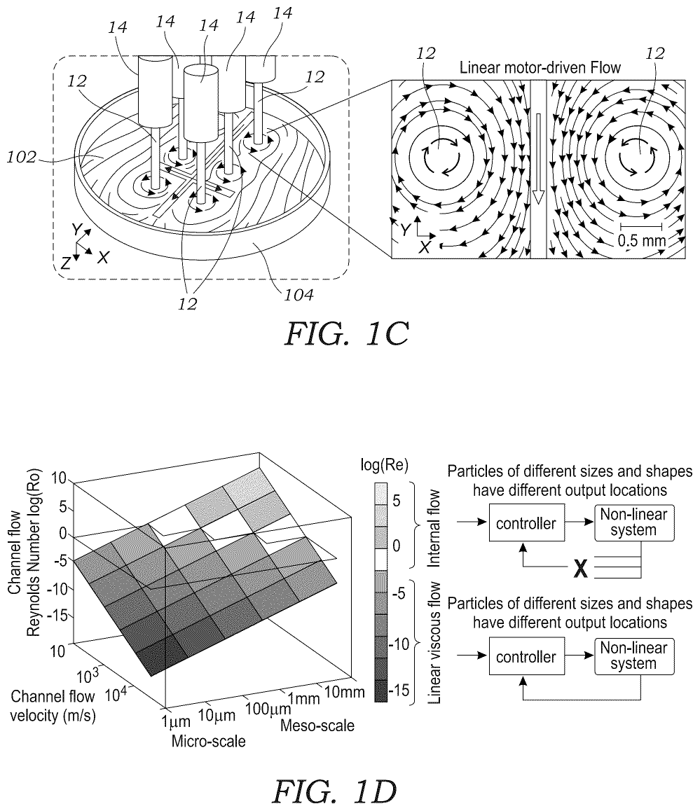

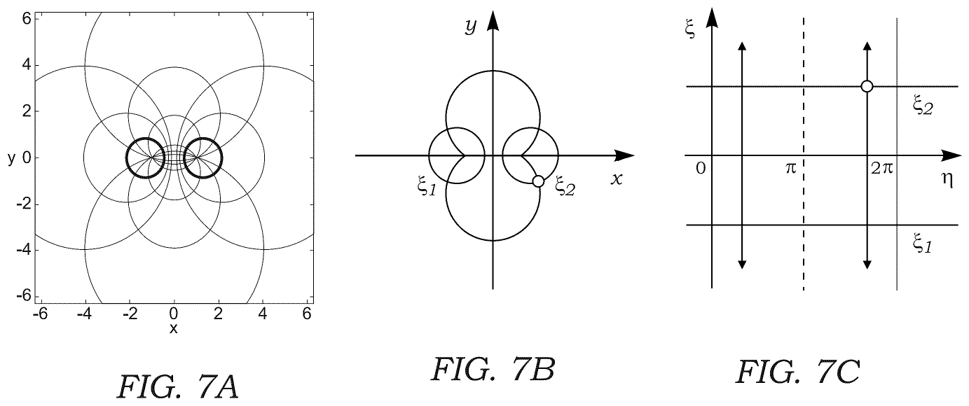

To drive linear fluid flow between motors 14, an investigation was first done on flow induced by the counter rotation of two co-axial circular motors immersed in stagnant fluid (FIG. 2A) that acted as the rotatable shafts 12. Assuming two circular shafts with the same radii (a=a₁=a₂) are rotating in opposite sense (clockwise=Ω₁, anticlockwise=−Ω₂) at the same angular velocity (Ω=Ω₁=Ω₂), the flow Reynolds number is (Re=ua/v), where u is the motor wall velocity (u=Ω a) and v the kinematic viscosity. At low Re, Jeffery's solution is used to analytically solve for Stokes flow between the motor shafts and to validate the numerical model under viscous-dominated conditions (FIG. 2B, FIGS. 7A-7C). At the motor shaft wall-fluid interface, the motion of the motor shaft wall drives the rotation of the surrounding fluid 102. The flow driven by counter-rotating motors joins in the mid-line upstream of the two motors and separates downstream leading to the presence of two saddle points when approaching and receding from the motors, respectively. In between the motors 14, the flow is symmetric about the centerline and the counter rotating flow moves the fluid from the upstream saddle point passing through the centerline to the downstream saddle point. In between the saddle points, closed streamlines are formed around the two rotating motor shafts limiting the entry of far field fluid flow between the motors. This enables driving the flow unidirectionally in between the rotatable shafts 12 (i.e., motor shafts) in a manner that resembles flow in a straight microfluidic channel, however, without confinement. When Re is small, and particle inertia is negligible, the motion of objects 100, has no memory and therefore suspended mesoscale objects 100 of different sizes and shapes respond to follow flow streamlines and faithfully retrace previous positions when reversing the flow thus, enabling linear flow control.

### Mesoscale Motor-Driven Flow Control

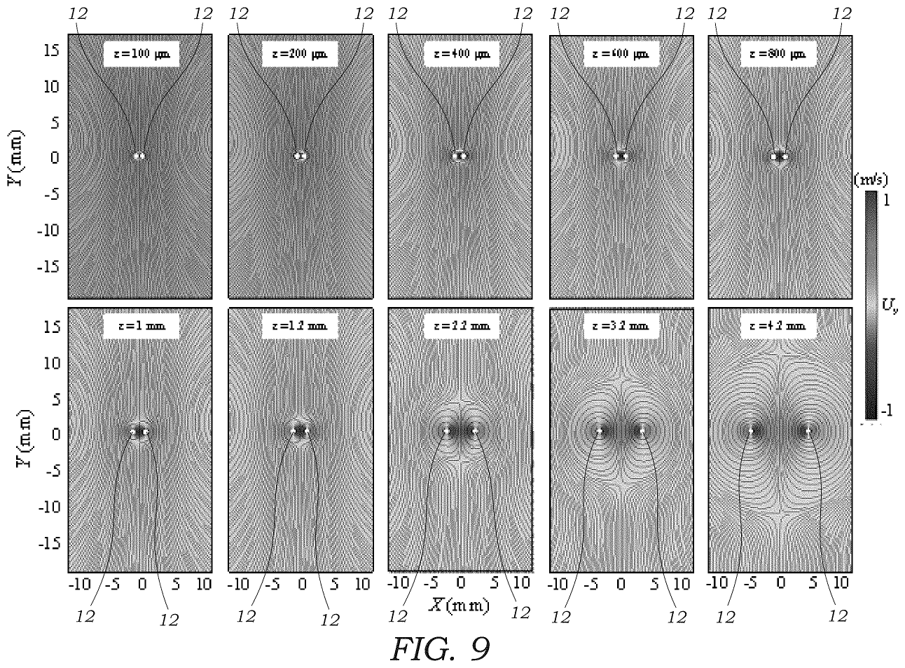

Next, to control mesoscale particles (i.e., objects 100) over a wide size range in counter-rotating motor shaft flow, the flow profile was studied as a function of the gap between the two motors shafts (ε=L−a) at the same low Re, where L is the distance between the centerline and the motor shaft centroid (FIG. 2A). The numerical simulation of the upwards flow velocity about the mid-plane between the motor shafts 12 matches the analytical solution of Jeffery's flow across a range of gaps, ε, from 100 μm to 2 mm (FIG. 2C). The two saddle points on the y-axis approach each other as ε decreases (FIG. 2D). The gap distance ε between the motor shaft walls determines the flow travel distance between the saddle points upstream (flow entry) and downstream (flow exit) of the motors mirroring the length of a microfluidic channel between the inlet and outlet. More importantly, ε also determines the flow width to fit a wide range of both micrometer scale (FIG. 2E) and millimeter scale (FIG. 2F) particles. In contrast to microfluidic technology, motor-driven flow is not predefined nor geometrically constrained by microfabrication or 3D printing. Therefore, motor-driven flow allows modularity to fit mesoscale objects 100 with a wide size range (i.e., resembling a modular channel width) to travel various distances (i.e., resembling a modular channel length) by simply changing the gap between the rotating shafts 12 (FIG. 9). This enables controlling flow in any fluid holder 104 (e.g., Petri dish) to match many functions of existing microfluidics.

### Scaling Linear Motor-Driven Flow Control

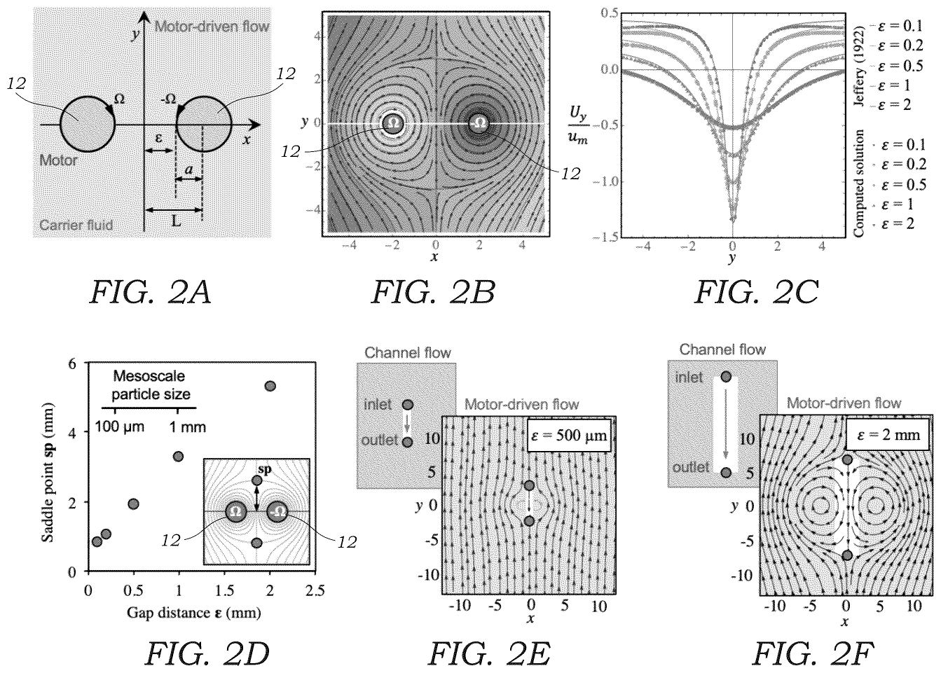

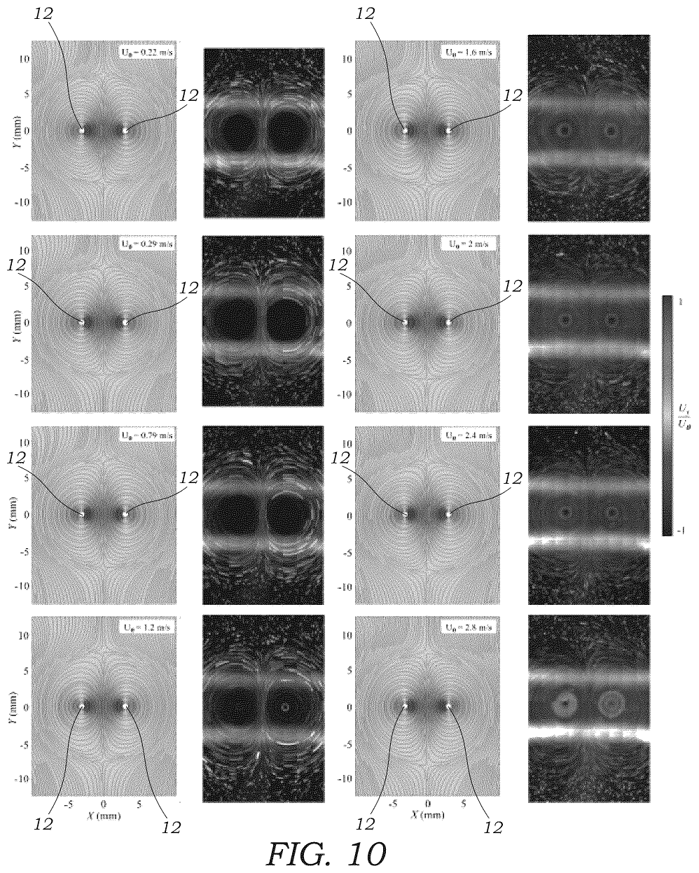

Next, it was explored how the results for Stokes flow extended to flows with finite inertia, to determine maximum operating conditions to scale manipulations. Motor-driven flow was investigated at a wider range of Reynolds number (Re=0.05−100) using numerical and experimental studies. The flow field was imaged and analyzed as a function of the Re and the characteristic length of the gap (h=a/L=0.1−0.9) between two stepper motors 14. Thus, enabling universal scaling and analysis of motor-driven flow for any motor size, gap distance, angular velocity, and carrier fluid properties. Numerical and experimental observations identified three motor-driven flow profiles summarized in a phase diagram (FIG. 3A). At Re<5 regardless the of the gap (h), the flow resembled Jeffery's flow—marked (◯) in the first row of FIG. 3B. The flow is symmetric about the centerline as demonstrated in the velocity field (FIG. 3C) and the pressure field (FIG. 3D). Saddle points were observed via fluorescent particle tracing as the flow is pushed out from both upstream and downstream saddle points which are symmetric (FIG. 10). This flow symmetry allows predictable and precise flow control upstream, downstream and between the motor shafts, which enables expanding flow control and manipulation through an ensemble of motor units (FIG. 3E).

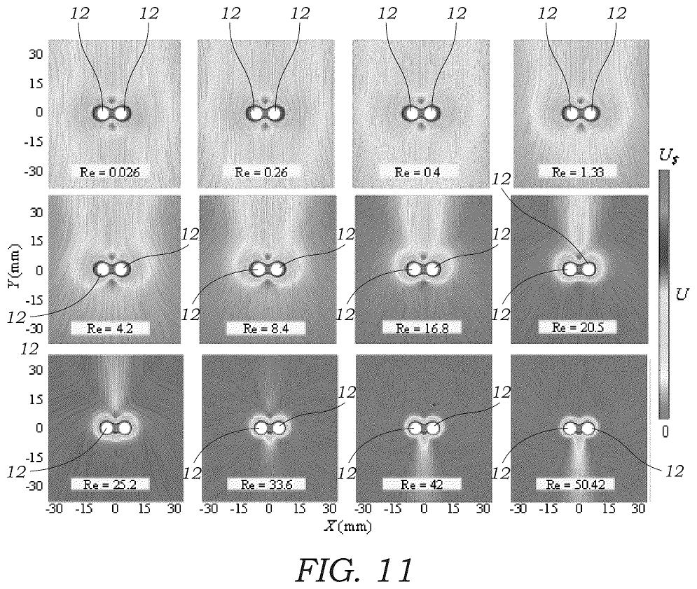

At higher Re, with motor shaft walls in close proximity (h>0.5) marked (Δ), flow is driven upward upstream of the motor shafts but loses this symmetry downstream of the motors (FIG. 3B-3E middle row). At even higher Re (·) regardless of the motor shaft proximity, a downstream flow occurs both upstream and downstream of the motor shafts, while also maintaining asymmetry (FIG. 3B-3E last row). These asymmetric flow topologies in Δ and · prevent adding upstream and downstream motor-units to form and connect unidirectional flow in between multiple motor shafts. Thus, these flow conditions limit scaling flow control in between a larger ensemble of motor shafts (FIG. 3E and FIG. 11). Because of this the symmetric flow ◯ at Re<5 and motor shaft distance h<0.25 was used, for arrays of upstream and downstream motor shafts to enable control of flow and hand-offs of objects 100 between multiple motor shafts (FIG. 3E). The optimal linear and reversible motor flow condition allows for precise flow control in between an ensemble of rotating shafts 12 in unconfined conditions.

Flow Control with Ensembles of Spatially Distributed Motors

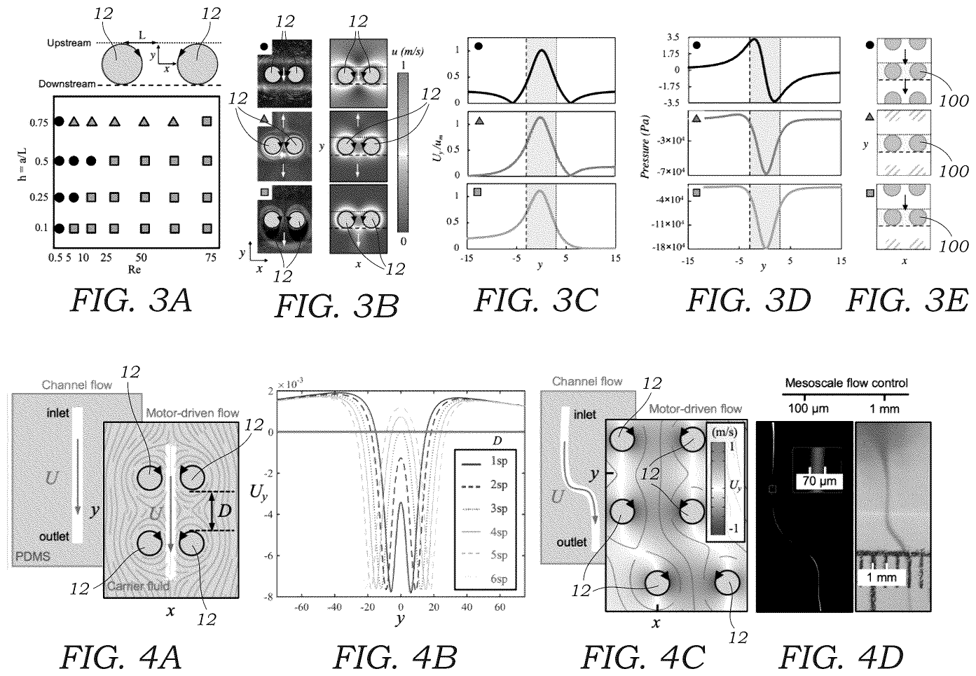

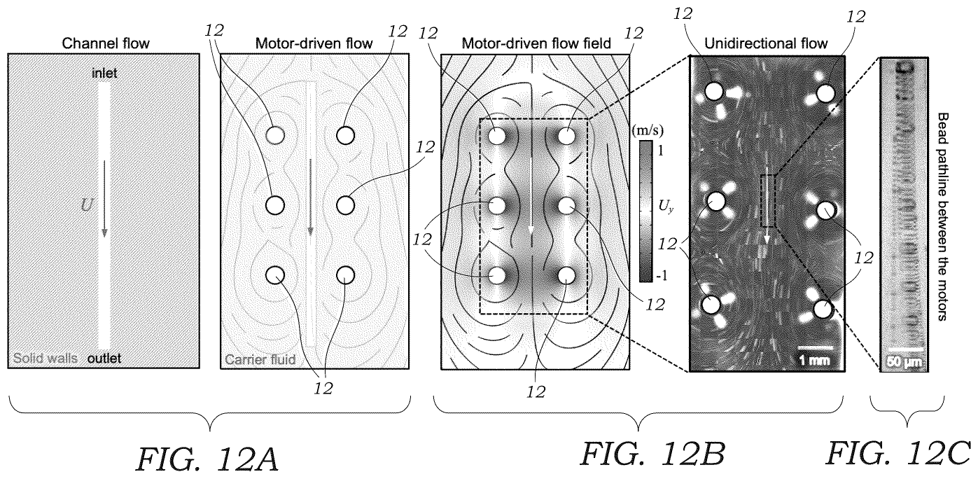

Using the positioning and rotation of an ensemble of rotatable shafts 12 in a Petri dish basic microfluidic-like functions were achieved (FIG. 4A). Since the saddle points are the limiting factor in the object 100 transport between multiple downstream rotatable shafts 12, the closed streamlines produced at a range of lateral gap distances (D) between neighboring rotatable shaft 12 pairs was investigated. Based on previous findings of closed streamline profiles in FIG. 2D and FIGS. 3A-3E, D was set as a function of the saddle point distance (sp) (FIG. 4B). It was found that 3sp is the largest gap distance to maintain connected unidirectional flow between pairs of rotatable shafts 12. This maximum distance (3sp) was used to reproduce precise unidirectional channel flow using multiple motor-units aligned in one direction (FIGS. 12A-12C). To change the channel flow shape or directions, the motor ensemble geometry was rearranged to drive the sample flow through different paths between the motor shafts (FIG. 4C). This enabled the precise control of the shape of a bundle of fluid streamlines along a directed paths between the motor shafts at millimeter length scales down to a length scale of 70 μm (FIG. 4D). Therefore, an ensemble of micro-motor shaft pairs was able to define pseudo microfluidic channels with varying geometry.

### Programmable Mesoscale Flow Control

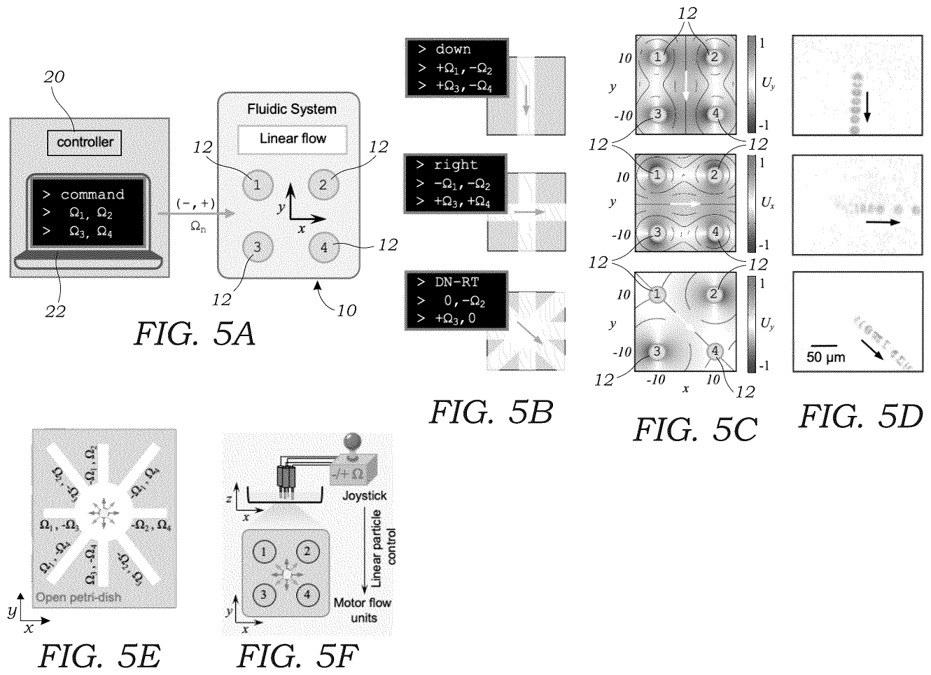

Next, the ability to manipulate and sort suspended objects 100 was investigated by changing the rotation orientation of an ensemble of rotatable shafts 12 in a quadrupole arrangement. A computer controller 20 was set to actuate specific motors 14 and their rotation orientation to move the objects 100 in different directions (FIG. 5A). This arrangement was able to achieve modular object manipulation in vertical (FIG. 5B-5D, first row), horizontal (FIG. 5B-5D, second row) and diagonal directions (FIG. 5B-5D, third row) by programming the rotation orientation (clockwise=Ω, anticlockwise=−Ω) in selected motors 14 (Ω₁, Ω₂, Ω₃, and Ω₄). In addition, the configuration enabled the transportation of an object 100 followed by the complete reversal of the object's motion to place it back at its original location since the device 10 is operating in linear Stokes flow viscous regime, with negligible inertial forces. This linear flow control property is essential to achieve simplified robotically-controlled motion that are only dependent on selected initial conditions. This enabled the control of object 100 (i.e., particle) motion along any available path defined by a joystick input 24, bypassing the use of multiple pumps to achieve the same flow control modularity in microfluidic devices (FIG. 5E). The device 10 was able to sort microparticle objects 100 in eight (8) different directions based on activated motor rotation orientation and angular velocity programmed in response to the input of the joystick 24 (FIG. 5F). To illustrate the accuracy of the linear control system, the joystick 24 was able to move objects 100 such as particles, cells and cell clusters along certain paths and then reverse the particle motion back to its original location. Therefore motor-driven flow can produce real-time linear, precise, and predictable spatiotemporal meso-scale flow control in-a-dish without the use of external pressure pumps, clean room fabrication or 3D printing.

### Automated Mesoscale Particle Sorting Via Closed-Loop Control

To automate mesoscale particle sorting and analysis, imaging and image analysis operations were integrated with fluidic operations controlled by motor-driven flow to achieve a closed-loop system. An image analysis code was executed in the computer controller 20 connected with an imaging device 30 (i.e., microscope camera) to recognize and acquire real-time locations of mesoscale objects 100 (i.e., particles) and count the number of cells within a particle. This image-based feedback system establishes a closed-loop control between the motor-driven flow system and the computer controller 20 (FIG. 6A). To illustrate the modularity and robustness of the automated mesofluidic system, droplets with different sizes and shapes were used for automated sorting and analysis. As a proof of concept, the system weas programmed to automatically sort based on the number of microalgae cells encapsulated per droplet. This enables selecting droplets with single cells for clonal analysis, resolving challenges with loading that is governed by Poisson statistics, resulting in numerous undesired empty droplets or droplets containing multiple cells. The droplet spatial coordinates and encapsulated cell number is established once the droplet is recognized by the computer controller 20 (FIG. 6B). Then the system continuously calibrates the speed and rotation orientation of the motors 14 to perform the following tasks (1) localize the droplet at the center of the motor quadrupole, (2) count cell number while rotating the droplet to get accurate cell number across different rotational angles, and (3) sort the droplet based on the cell number regardless of the droplet size or shape (FIGS. 6C-6D). The linear relation between motor rotation and fluid motion enables proportional control of suspended mesoscale droplet movement. Droplets of different sizes and structures were transported with the same motor actuation. In this way, cells encapsulated in droplets were able to be sorted with the same response time, as the differences in size and shape of droplets results in negligible differences in inertia. With the linear closed-loop system control, sorting and analysis of droplets containing microalgae was automated achieving 86% accuracy in isolating droplets with 0, 1 or 2 microalgae to separate outlets with a low cost ($200) computer controller 20 and integrated motor-driven flow.

A motor-driven flow and particle manipulation technology is disclosed based on the analytical solution of two counter rotating cylinders, known as Jeffery's flow. This flow is extended to a programmable ensemble of semi-suspended robotic motors 14 with rotatable shafts 12 (i.e., motor shafts) within an unconfined Petri dish, coupled with linear closed-loop control via a controller 20. The device 10 achieves precise linear control of mesoscale particle movement at high velocities that would otherwise produce inertial and non-linear responses in confined channel flow (FIG. 1D). A synchronized ensemble of motors 14 reproduced basic flow functions from microfluidic channel flow (e.g., translate, rotate, and separate suspended particles) while maintaining linear flow control and bypassing the need for new chip designs when a new channel topology is needed. Using the linear system behavior, particle analysis and movement were automated by integrating imaging, machine vision and linear feedback control. This accessible, modular, and low-cost motor-driven flow technology can automate mesoscale particle analysis and sorting and pave the way for future applications of AI-driven robotic research workflows.

### Materials and Methods

### Theoretical Framework of Motor-Driven Flow

To analyze motor-flow control via numerical and experimental methods, the motor-driven flow model was validated at low Reynolds number conditions with Jefferey's analytical solution. Since the height of the rotatable shaft 12 of the motor 14 is much larger than the size of the object 100 (i.e., particle) in flow, it was assumed the rotatable shafts 12 are of infinite height to simplify to a 2-dimensional linear stokes approximation:

$$μ {∇}^{2} u = ∇ p \qquad ( 1 )$$

$$∇ \cdot u = 0 \qquad ( 2 )$$

with u, ρ, μ representing the velocity, pressure, and dynamic viscosity of the fluid respectively. The flow between two identical counter rotating circular cylinders or motors (i.e., shafts 12) suspended in fluid are well adapted to be solved in bipolar coordinate system as it offers the unique use of surface constants corresponding to cylinders radii, and the symmetry presented in them (FIG. 7A). The solution of the streamfunction biharmonic equation ∇⁴ψ=0 in bipolar coordinates via conformal mapping is in the form

$$ζ ≡ ξ + i η = log \frac{z + c}{z - c} \qquad ( 3 )$$

$$with$$

$$z ≡ x + iy = log \frac{x + i ( y + a )}{x + i ( y - a )}$$

where, the quasi-radial coordinates (ξ) corresponds to the coaxial circles with centers along the x-axis and range −∞<ξ<+∞. The angular coordinates (17) corresponds to interesting circles with centers along the y-axis and range 0<η<2π (FIGS. 7B and 7C):

$$ξ ( x , y ) = \frac{1}{2} log \frac{{( x + c )}^{2} + {y}^{2}}{{( x - c )}^{2} + {y}^{2}} , η ( x , y ) = {tan}^{- 1} [ \frac{x + c}{y} ] - {tan}^{- 1} [ \frac{x - c}{y} ] \qquad ( 4 )$$

$$and scale factor h ( ξ , η ) = \frac{cos h ξ - cos η}{c} \qquad ( 5 )$$

ξ=ξ₁, ξ₂ was set to represent the motor cylinder walls with radius (r=c cosech|α|), center (C=c coth|α|), and gap distance (ε=cosh|α|−1) along the x-axis (darker circles) (FIG. 7A). The streamfunction (ψ) for counter rotating cylinders defined by Jeffery 1922 (FIG. 2B):

$$h ψ ( x , y ) = {b}_{0} ξ ( cos h ξ - cos η ) + {c}_{0} sin h ξ + {c}_{1} sin h 2 ξcos η \qquad ( 6 )$$

with boundary conditions corresponds to motor's cylindrical wall rotation and a no slip boundary of the outer circular wall, the constants are determined by the following

$${b}_{0} = - \frac{cos h 2 α}{2 cos h 2 α sin {h}^{2} α} , \qquad ( 7 )$$

$${c}_{0} = \frac{cos h α}{2 sin {h}^{2} α} ,$$

$${c}_{1} = - \frac{1}{4 cos h α sin {h}^{2} α}$$

The motor flow profile and streamline function were computed and plotted using Mathematica and Julia programming language.

### Motor-Driven Flow Simulation

To investigate motor-driven flow in a wider range of Reynolds numbers (Re=0.5−100), the finite element computation (Matlab and COMSOL Multiphysics 5.2) was first validated using the theoretical or analytical solution provided in the previous section. An incompressible fluid in steady state laminar flow was used in the computational model based on Navier-Stokes equation:

$$ρ ( \frac{\partial u}{\partial t} + u \cdot ∇ u ) = - ∇ p + μ {∇}^{2} u \qquad ( 8 )$$

$$∇ \cdot u = 0 , \qquad ( 9 )$$

with u, p, ρ and μ representing the velocity, pressure, density, and carrier fluid dynamic viscosity of the fluid respectively. No-slip boundary conditions are applied on the fluid container walls and slip condition with angular velocity (U_{θ}) is applied on the walls of the rotatable shaft 12. Similar to the analytical solution, the reference point (x₀, y₀) of the two-dimensional model is centered in the midline (y=0) between the rotatable shafts 12 with gap (ε=cosh|α|−1). Here, intuitive representation of two-dimensional motor-flow cross section was intended to provide a general understanding of the flow control system and enable researchers to develop novel designs for their mesoscale particle manipulation systems. In such set up, the heat color map shows positive values in close proximity to the rotatable shafts 12 in FIG. 3Band a negative values further away from rotatable shafts 12 in FIG. 3B of the velocity components (Uₓ and U_{y}), and to illustrate the rotation orientation (i.e., clockwise=Ω₁, anticlockwise=−Ω₂) of each motor. However, detailed simulations can be found FIGS. 7A-7C to define the motor flow behavior transitions in different regimes. The velocity profile of the carrier fluid (u) is normalized by motor angular velocity (U_{θ}) and plotted as a function of the y-axis in FIG. 3C.

### Motor-Flow Field Experimentation

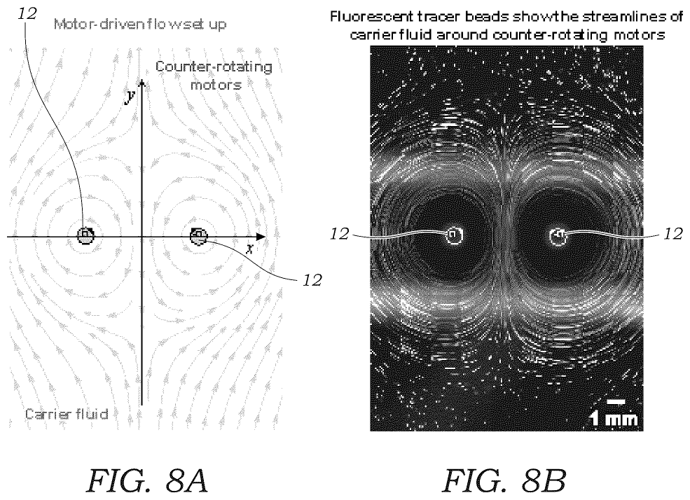

To investigate motor-driven flow in unconfined conditions, stepper motor shafts were used as the rotatable shafts 12 in an open Petri dish that acted as the fluid holder 104. The coreless stepper micromotors 14 are powered by DC voltage and the shaft angular velocity is controlled by the voltage input. Different input voltages were tested and rotation of the motor via highspeed camera was observed to calculate angular velocity (UI) (Rotation Per Minute RPM) (FIG. 8A). Then the rotational velocity is converted to the linear velocity of the motor shafts (uₘ) (FIG. 8B). Motors and their respective motor shafts 12 are fixed on top of the fluid holder 104 at different distances. Glycerol carrier fluid is fluorescently labeled with rhodamine and fluorescein dyes (ThermoFisher Scientific) to track unidirectional flow between motors at the microscale. Fluorescent polystyrene particles with mean diameter 30 μm are purchased from (Phosphorex, Hopkinton, MA) were used to trace the flow profile around the rotatable shafts 12. At the milliscale food dye was used to track different flow designs between the rotatable shafts 12 (e.g., motor shafts). To investigate motor-flow behaviors, the motor flow profile was imaged at different cylindrical motor shafts diameters, gap distance, angular velocity, and carrier fluid viscosity (FIG. 3A).

### Imaging

Flow between the rotatable shafts 12 of the motors 14 (i.e., motor shafts) was viewed using a microscope 30 (Nikon Ti-U) connected to high-speed Phantom V2010 camera (Vision Research Inc., Wayne, NJ, USA). Images were recorded at 1000 frames per second for characterizing motor flow velocity between motors. Image processing code was developed with MATLAB to identify particle location and track particle motion. The fluorescence images are captured by CCD Coolsnap HQ2 camera (Roper Scientific, Evry, France). Particle tracers are imaged at 2 frames per second for analyzing the behavior of carrier fluid flow between motors.

### Automated Mesofluid Manipulation

The image analysis code was connected to the motor ensemble controller 20 to actuate the motor rotation via continuous feedback control. As a proof of concept, image recognition via OpenCV library in a raspberry pi setup was used to automate manipulation of suspended particles, cells and droplets. MCF-7 cells and cell clusters (30 2004, ATCC, Manassas, VA) were cultured in DMEM medium (ATCC 30-2002). The medium was supplemented with 10% fetal bovine serum (Invitrogen, Carlsbad, CA) and 1% penicillin streptomycin (Invitrogen, Carlsbad, CA) at 37 C under 5% CO2 conditions. Cells were passaged once they reached an 80% confluence. The cells and medium tested negative for mycoplasma. Droplets are made by step emulsification and introduced to the system by pipetting into the carrier fluid at a location upstream of the motors. Droplet formation and cell encapsulation methods are described in detail in M. Li et al., “A Gelatin Microdroplet Platform for High-Throughput Sorting of Hyperproducing Single-Cell-Derived Microalgal Clones,” Small, vol. 14, no. 44, Nov. 2018, which is incorporated by reference. Image analysis and direct feedback control were programmed to automate the sorting of algae based on the cell number per encapsulated droplet, and sorted them down when cell count=0 cells, right for 1 cell, and left for 2 cells.

### Image-Based Closed Loop Control Code

The following is the image analysis and tracking code that was used with closed-loop particle manipulation and control:

```
# Import the necessary packages
from picamera.array import PiRGBArray
from picamera import PiCamera
import time
import cv2
import numpy as np
import serial
import threading
# Initialize the camera and grab a reference to the raw camera capture
camera = PiCamera( )
camera.resolution = (640, 480)
camera.framerate = 30
rawCapture = PiRGBArray(camera, size=(640, 480))
singleCapture = PiRGBArray(camera, size=(1640, 1232))
norm_mask = None
have_droplet_in_ROI = False
# Initialize arduino serial
# arduinoSerialData = serial.Serial(‘/dev/ttyACM0’,9600)
# Allow the camera to warmup
time.sleep(0.1)
img_num = 0
def sort_single_droplet(orig_x,orig_y,orig_r,single):
  x = int(2.56*orig_x) − (1664/2 − int(2.56*60))
  y = int(2.56*orig_y) − (1232/2 − int(2.56*60))
  r = int(2.56*orig_r)
  mask = np.zeros((2*int(2.56*60),2*int(2.56*60)), np.uint8)
  gray_single = cv2.cvtColor(single,cv2.COLOR_BGR2GRAY)
  # Contour Detection
  # A circular mask is placed on the original gray image to eliminate
  # any pixels that are not directly within the detected droplet. Thresholding
  # is performed by allowing the darkest 5% of pixels within the droplet to remain
  # since the cells are typically the darkest part of the droplet.
  print gray_single.shape
  print mask.shape
  cv2.circle(mask,(x,y),r−5,(255,255,255),−1)
  local_image = cv2.bitwise and(gray_single,gray_single,mask = mask)
  val_array = [a for a in local_image flatten( ) if a > 0]
  retval, thresh_local_image = cv2.threshold(local_image, np.percentile(val_array, 5), 255,
cv2.THRESH_TOZERO_INV)
  thresh_local_image = cv2.GaussianBlur(thresh_local_image, (3,3), 0)
  img, contours, hierarchy =
cv2.findContours(thresh_local_image,cv2.RETR_TREE,cv2.CHAIN_APPROX_SIMPLE)
  # Ellipse Fitting
  # An ellipse is fit to every valid contour under the assumption that each
  # valid contour is a cell with a roughly oval shape.
  num_cells = 0
  for contour in contours:
    if len(contour) > 5:
      num_cells = num_cells + 1
      el = cv2.fitEllipse(contour)
      cv2.ellipse(single,el,(0,255,0),2)
  cv2.putText(single,“{0} cells”.format(num_cells),(x−30,y−70), 1, 2, (120,0,255),1,cv2.LINE_AA)
  # Command the Arduino through a Serial connection
  # The command syntax is: “(direction)*(milliseconds)*(speed)”
  # For example: down*1000*10 is direction down for 1 second at speed 10.
  if num_cells == 0;
    cv2.putText(single,“DOWN”,(10,10), 1, 1, (0,0,255),1,cv2.LINE_AA)
## arduinoSerialData.write(“down*1000*50”)
  elif num_cells == 1:
    cv2.putText(single,“RIGHT”,(10,10), 1, 1, (0,0,255),1,cv2.LINE_AA)
## arduinoSerialData.write(“right*1000*50”)
  else:
    cv2.putText(single,“LEFT”,(10,10), 1, 1, (0,0,255),1,cv2.LINE_AA)
## arduinoSerialData.write(“left*1000*50”)
  # Adjust global variable to allow another droplet to be sorted
  global have_droplet_in_ROI
  have_droplet_in_ROI = False
  cv2.imshow(“Processed Single Shot”, single)
  cv2.imwrite(“IMG_1.png”, single)
# Capture frames from the camera
for frame in camera.capture_continuous(rawCapture, format=“bgr”, use_video_port=True):
  image = frame.array
  # Rotate image
      # (h,w) = image.shape[:2]
      # center = (w/2,h/2)
      # rot_mat = cv2.getRotationMatrix2D(center, 180, 1.0)
      # image = cv2.warpAffine(image, rot_mat, (w,h))
  gray_image = cv2.cvtColor(image,cv2.COLOR_BGR2GRAY)
  # Wait for the whole frame time for a key to be pressed and perform
  # bitwise AND operation to retrieve ord data for the key press.
  key = cv2.waitKey(1) & 0xFF
  # Background Normalization
  # Taking the median of the background image and creating a mask that
  # adjusts a true image to erase background features.
  if key == ord(“b”):
    median = int(np.median(gray_image))
    norm_mask = median − gray_image
  if norm_mask is not None:
    gray_image = gray_image + norm_mask
  # Image Thresholding
  # Adaptive image thresholding is performed on a gaussian blurred image
  # to extract distinct droplet outlines. All parameters determined by
  # “brute force learning” i.e. looping through possible values.
  adj_image = cv2.GaussianBlur(gray_image, (3,3), 0)
  adj_image =
cv2.adaptiveThreshold(adj_image,255,cv2.ADAPTIVE_THRESH_GAUSSIAN_C,cv2.THRESH_BI
NARY,9,2)
  circles = cv2.HoughCircles(adj_image,cv2.HOUGH_GRADIENT, 1.2, 100.0, param1=50,
param2=25, minRadius=20, maxRadius=30)
  if circles is not None:
    circles = np.round(circles[0, :]).astype(“int”)
    circles_array = np.array(circles)
    for circle_num,(x,y,r) in enumerate(circles):
      cv2.circle(image,(x,y),r,(0,255,0),4)
      cv2.putText(image, “X:{0} Y:{1} r:{2}”.format(x,y,r),(x−30,y+50), 1, 0.5,
(0,0,255),1,cv2.LINE_AA)
      cv2.putText(image,“{0}px”.format(r),(x−30,y−50), 1, 2, (120,0,255),1,cv2.LINE_AA)
      # For a droplet within the ROI
      if (x > 320−60+r) and (x < 320+60−r) and (y > 240−60+r) and (y < 240+60−r):
        cv2.circle(image,(x,y),r,(255,255,0),4)
        if not have_droplet_in_ROI:
          # This prevents the code from continuing to take pictures
          # of the same droplet while it is being sorted
          have_droplet_in_ROI = True
          # Take single image at 2.5625 times resolution of 640x480
          camera.resolution = (1664, 1232)
          camera.capture(singleCapture, format=“bgr”)
          single = singleCapture.array
          camera.resolution = (640, 480)
          #Running the contour image analysis asynchronously by utilizing threading.
          #sort_single_droplet(x,y,r,single[(1232/2 − int(2.56*60)):(1232/2 + int(2.56*60)),(1664/2
− int(2.56*60)):(1664/2 + int(2.56*60))])
```

| | |
|---|---|
| ## | print “Making thread...” |
| ## | t= |

threading.Thread(target=sort_single_droplet,name=“Sorting”,args=(x,y,r,single[(1232/2 −
int(2.56*60)):(1232/2 + int(2.56*60),(1664/2 − int(2.56*60):(1664/2 + int(2.56*60))]))

| | |
|---|---|
| ## | t.daemon = True |
| ## | t.start( ) |

```
  if key == ord(“z”):
    cv2.imwrite(“test.png”, gray_image)
    print “SAVED IMAGE”
  # Actions performed to original frame before display
  cv2.rectangle(image,(320−60,240−60),(320+60,240+60),(0,128,100),4)
  cv2.imshow(“Frame”, image)
  # Clear the stream in preparation for the next frame
  rawCapture.truncate(0)
  singleCapture.truncate(0)
```

While embodiments of the present invention have been shown and described, various modifications may be made without departing from the scope of the present invention. For example, while droplet objects 100 were illustrated, the objects may include non-droplet objects. Objects 100 may include cells, beads, clusters of cells, organoids, spheroids, or other particles and not contained in a droplet or emulsion. The invention, therefore, should not be limited, except to the following claims, and their equivalents.

1. A device for the control of objects in a fluid within unconfined conditions, the device comprising:
a plurality of rotatable shafts arranged in an array or pattern and in physical contact with the fluid containing the objects, the plurality of rotatable shafts coupled to respective motors; and
a controller or computing device operatively coupled with each of the respective motors and configured to control one or more of a rotational direction and a rotational speed of the plurality of rotatable shafts.

2. The device of claim 1, wherein the objects range in size from ˜100 μm to ˜1 mm.

3. The device of claim 1, further comprising an imaging device configured to image the objects in the fluid.

4. The device of claim 3, further comprising image analysis software configured to track the spatial location of the objects in the fluid.

5. The device of claim 4, further comprising a trained neural network or algorithm configured to identify a class or type of object based on the image(s) of the object in the fluid.

6. The device of claim 1, wherein the plurality of rotatable shafts comprise at least four (4) rotatable shafts arranged in a quadrupole configuration.

7. The device of claim 1, further comprising an input device operably coupled to the controller and configured to control the rotational speed and/or the rotational direction of the plurality of rotatable shafts in response to manipulation of the input device.

8. The device of claim 1, wherein the plurality of rotatable shafts are located in a container, dish, or well.

9. The device of claim 1, wherein the objects are contained in droplets.

10. The device of claim 1, wherein the objects comprise droplets, beads, cells, spheroids, organoids, or cellular structures.

11. A method of manipulating objects in a fluid within unconfined conditions comprising:
providing a plurality of motor-driven rotatable shafts in contact with the fluid containing the objects; and
controlling a rotational speed and/or a direction of the plurality of rotatable shafts to controllably manipulate the objects within the fluid.

12. The method of claim 11, wherein the objects are moved to one or more target locations.

13. The method of claim 11, further comprising imaging the objects within the fluid to identify one or more specific types or classes of objects and wherein the objects are moved to one or more target locations based on the identified one or more specific types or classes of the objects.

14. The method of claim 13, wherein the identity of one or more specific types or classes of objects is automatically performed by image analysis software.

15. The method of claim 11, wherein the objects comprise droplets, beads, cells, spheroids, organoids, or cellular constructs.

16. The method of claim 15, wherein the cells or cellular constructs are contained in the droplets.

17. The method of claim 11, wherein at least one of the plurality of rotatable shafts drives a flow within a fluid.

18. The method of claim 11, wherein at least two of the plurality of rotatable shafts drives a flow within a fluid.

19. The method of claim 11, wherein the plurality of rotatable shafts comprise at least four (4) rotatable shafts arranged in a quadrupole configuration.

20. The method of claim 17, wherein the flow within the fluid is at a Reynolds number of less than 10.

21. The method of claim 17, wherein the flow within the fluid is at a Reynolds number of less than 5.

22. The method of claim 11, wherein at least two of the plurality of rotatable shafts rotate in opposing directions.
